(19) United States
(12) Patent Application Publication
(10) Pub. No.: US 2024/0066085 A1
(43) Pub. Date: Feb. 29, 2024

(54) MATRIX METALLOPROTEASE 1 EXPRESSION INHIBITOR, SKIN EXTERNAL AGENT, AND USE FOR INHIBITION OF MATRIX METALLOPROTEASE 1 EXPRESSION

(71) Applicants: NATIONAL UNIVERSITY CORPORATION KOBE UNIVERSITY, Hyogo (JP); DSR CORPORATION, Hyogo (JP)

(72) Inventors: Yasuhito SHIRAI, Hyogo (JP); Koji TAKAOKA, Hyogo (JP)

(73) Assignees: NATIONAL UNIVERSITY CORPORATION KOBE UNIVERSITY, Hyogo (JP); DSR CORPORATION, Hyogo (JP)

(21) Appl. No.: 18/033,964

(22) PCT Filed: Oct. 26, 2021
(86) PCT No.: PCT/JP2021/039566

§ 371 Date: Nov. 3, 2023

(30) Foreign Application Priority Data
Oct. 28, 2020 (JP) 2020-180871

(51) Int. Cl. A61K 36/185; A61K 9/00; A61K 36/484; A61K 36/53; A61K 36/537

(52) U.S. Cl. CPC A61K 36/185; A61K 9/0014; A61K 36/484; A61K 36/53; A61K 36/537

(57) ABSTRACT

Expression of matrix metalloproteinase 1 is suppressed. A matrix metalloproteinase 1 expression suppression agent of this disclosure contains, as an active ingredient, at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop.

### TECHNICAL FIELD

The present disclosure relates to a matrix metalloproteinase 1 expression suppression agent, a skin external agent, and use for suppression of matrix metalloproteinase 1 expression. This application claims priority on Japanese Patent Application No. 2020-180871 filed on Oct. 28, 2020, the entire content of which is incorporated herein by reference.

### BACKGROUND ART

Formation of wrinkles is due to degradation of hyaluronic acid or collagen in the dermis. PATENT LITERATURES 1 and 2 each disclose a skin care technology regarding this point. Specifically, PATENT LITERATURE 1 discloses use of NEI-L1 being a neutrophil elastase suppression component, in order to eliminate skin problems. NEI-L1 penetrates the dermis and acts on neutrophils, to suppress elastase generated by neutrophils.

PATENT LITERATURE 2 discloses application of retinol to a skin cosmetic. Retinol acts on keratinocytes to cause a hyaluronic acid synthesis promotion effect to appear, and exhibits a wrinkle improvement effect due to an effect of increasing the moisture in the stratum corneum.

### CITATION LIST

### Patent Literature

PATENT LITERATURE 1: Japanese Laid-Open Patent Publication No. 2019-022483
PATENT LITERATURE 2: Japanese Laid-Open Patent Publication No. 2013-189397

### Non Patent Literature

NON PATENT LITERATURE 1: Olga S, Michael V, Michael N. (2012) BMJ Journal. 62, 358-367.

### SUMMARY OF INVENTION

### Technical Problem

As described above, formation of wrinkles is largely due to degradation of collagen. However, to date, suppression of formation of wrinkles, by focusing on MMP-1 being an enzyme that degrades collagen, has not been known. The present inventors found a novel technology that suppresses expression of MMP-1.

An aspect of the present disclosure is a matrix metalloproteinase 1 (MMP-1) expression suppression agent. The MMP-1 expression suppression agent of the disclosure contains, as an active ingredient, at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop. The present inventors experimentally found that each plant described above or an extract thereof suppresses expression of MMP-1.

The MMP-1 expression suppression agent of the disclosure suppresses expression of MMP-1, through suppression of activity of protein kinase C-delta (PKC6). NON PATENT LITERATURE 1 discloses that activity of protein kinase C (PKC) is involved in expression of MMP-1 due to ultraviolet rays. However, to date, for suppression of formation of wrinkles, no specific subtype of PKCδ has been focused on.

The present inventors experimentally clarified that, out of some subtypes of PKC that are expressed in the skin, PKCδ is involved in the expression pathway of MMP-1. Further, the present inventors clarified that the MMP-1 expression suppression agent suppresses expression of MMP-1, through inhibition of activity PKC6. The MMP-1 expression suppression agent can suppress expression of MMP-1, by acting on PKC6. In other words, PKC serves as a site of action, whereby expression of MMP-1 is suppressed. Thus, the MMP-1 expression suppression agent preferably contains, as an active ingredient, a PKCδ inhibitor. The MMP-1 expression suppression agent (PKC6 inhibitor) may indirectly inhibit activity of PKC6, by acting on the upstream side of PKCδ in the MMP-1 expression mechanism including PKC6.

Another aspect of the present disclosure is a skin external agent. The skin external agent of the disclosure contains the MMP-1 expression suppression agent. The skin external agent of the disclosure suppresses wrinkles of the skin, through suppression of expression of MMP-1.

Still another aspect of the present disclosure is use, for matrix metalloproteinase 1 expression suppression, of at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop. In this use, preferably, expression of matrix metalloproteinase 1 can be suppressed through inhibition of activity of protein kinase C-delta.

Still another aspect of the present disclosure is use, for production of a skin external agent for matrix metalloproteinase 1 expression suppression, of at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop. Preferably, the skin external agent is for suppressing expression of matrix metalloproteinase 1, through inhibition of activity of protein kinase C-delta.

Further details will be described later as an embodiment.

### BRIEF DESCRIPTION OF DRAWINGS

FIG. 1 is a schematic diagram of an MMP-1 expression mechanism.

FIG. 2 is a graph showing effects of plant extracts on UV-induced MMP-1 expression.

FIG. 3 shows effects of Saxifraga sarmentosa and hop on UV irradiation mice.

FIG. 4 shows effects of Saxifraga sarmentosa and hop on phosphorylation of PKC in UV irradiation mice.

FIG. 5A shows the change rate of the wrinkle area.

FIG. 5B shows the change rate of the maximum wrinkle depth.

### DESCRIPTION OF EMBODIMENTS

<1. MMP-1 Expression Mechanism>

MMP-1 is also referred to as interstitial collagenase, and is an enzyme that is involved in tissue destruction and tissue reconstruction. The expression amount of MMP-1 increases due to aging and ultraviolet ray exposure, and MMP-1 specifically cleaves the helix site of collagen in the dermis. Thus, MMP-1 is greatly involved in formation of wrinkles. Therefore, by suppressing expression of MMP-1, it is possible to suppress formation of wrinkles.

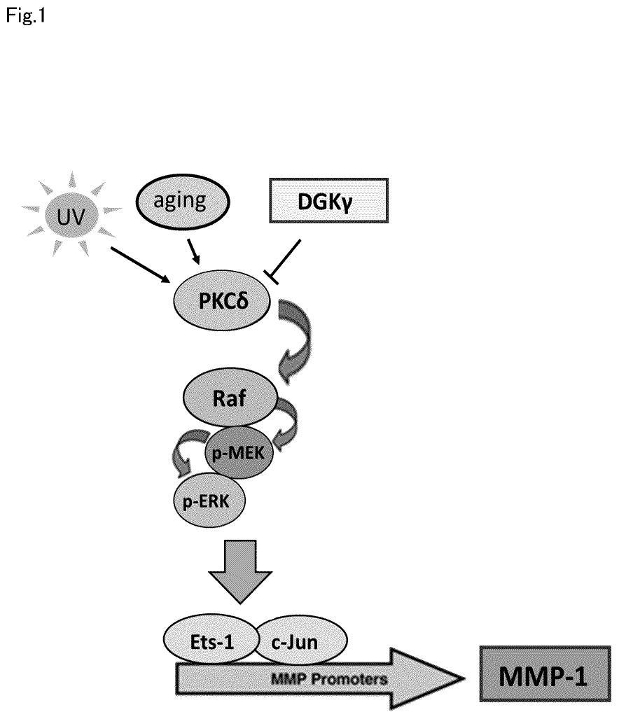

The present inventors clarified an expression mechanism of MMP-1 through an experiment. FIG. 1 shows the expression mechanism of MMP-1 clarified by the present inventors. The experiment was performed by irradiating HaCaT (epidermal keratinocyte cell line established from a human adult under special ca 2+ concentration and temperature conditions) with ultraviolet rays (UVA).

When UVA irradiation is performed on HaCaT, the MMP-1 expression amount increases in an irradiation-dependent manner. However, when a pretreatment of adding rotterlin being a PKCδ inhibitor to HaCaT was performed, and then UVA irradiation was performed on HaCaT, MMP-1 expression was significantly suppressed. Therefore, it was clarified that PKCδ is involved in expression pathway of MMP-1.

Further, when the expression amount of MMP-1 increased through UVA irradiation, ERK (extracellular signal-regulated kinase) and MEK (mitogen-activated protein kinase kinase) being proteins downstream of PKCδ also increased. However, when a pretreatment of adding rotterlin being a PKCδ inhibitor to HaCaT was performed, and then UVA irradiation was performed on HaCaT, induced expression of phosphorylation of ERK and MEK was suppressed. Therefore, it was clarified that: the activity of PKCδ induces phosphorylation of MEK and ERK downstream of PKC6; and expression of MMP-1 is promoted through c-Jun and the like.

According to the expression mechanism shown in FIG. 1, it is understood that, in order to suppress expression of MMP-1, controlling the activity of PKCδ so as to be negative is more effective. By controlling the activity of PKCδ so as to be negative, it is possible to effectively suppress expression of MMP-1 (formation of wrinkles) due to aging or ultraviolet rays.

<2. MMP-1 Expression Suppression Agent>

An MMP-1 expression suppression agent according to the embodiment contains, as an active ingredient, a plant described later or an extract thereof. As the plant, for example, at least one part selected from the group consisting of leaf, stem, root, flower, and fruit can be used. For example, the plant may be used, as an active ingredient, in a form of a ground product or a dried ground product of the plant itself.

The plant contained, as an active ingredient, in the MMP-1 expression suppression agent according to the embodiment is at least one selected from the group consisting of Saxifraga sarmentosa, rosemary, licorice, Melissa officinalis, wild thyme, and hop. These plants can be used for matrix metalloproteinase 1 expression suppression. Further, these plants can be used for production of a skin external agent for matrix metalloproteinase 1 expression suppression.

As an active ingredient for the MMP-1 expression suppression agent, an extract of each plant described above is suitably used. The extract is extracted from the plant into a solvent by using a known extraction method. The extract may be in a form of a liquid or a powder (dry powder). For production of the extract, at least one part selected from the group consisting of leaf, stem, root, flower, and fruit of the plant is used.

The extract contained, as an active ingredient, in the MMP-1 expression suppression agent according to the embodiment is at least one selected from the group consisting of an extract of Saxifraga sarmentosa, an extract of rosemary, an extract of licorice, an extract of Melissa officinalis, an extract of wild thyme, and an extract of hop. These extracts can be used for matrix metalloproteinase 1 expression suppression. Further, these extracts can be used for production of a skin external agent for matrix metalloproteinase 1 expression suppression.

Saxifraga sarmentosa is a plant of genus Saxifraga of family Saxifragaceae, and is an evergreen perennial herb. Rosemary is a plant of genus Rosmarinus of family Lamiaceae, and is an evergreen shrub. Licorice is a plant of genus Glycyrrhiza of family Fabaceae, and is a perennial herb. Melissa officinalis is a plant of genus Melissa and family Lamiaceae, and is a perennial herb. Melissa officinalis is also referred to as lemon balm or sweet balm. Wild thyme is a plant of genus Thymus of family Lamiaceae, and is a perennial plant. Wild thyme is also referred to as Thymus serpyllum. Hop is a plant of genus Humulus of family Cannabaceae, and is a climbing perennial herb.

Each plant described above or an extract thereof suppresses expression of MMP-1. That is, the plant described above or an extract thereof is suitable as an active ingredient of the MMP-1 expression suppression agent. The plant described above or an extract thereof suppresses formation of wrinkles, by suppressing expression of MMP-1. When the MMP-1 expression suppression agent suppresses expression of MMP-1 through inhibition of the activity of PKGδ, the MMP-1 expression suppression agent is also a PKCδ inhibitor.

The plant or an extract thereof itself may be used as the MMP-1 expression suppression agent, but may be contained, as an active ingredient, in a liquid or paste-like formulation, for example. The combination proportion of the plant or an extract thereof in the MMP-1 expression suppression agent is not limited in particular, and is preferably not lower than 0.01% by mass and not higher than 20% by mass, more preferably not lower than 0.1% by mass and not higher than 10% by mass, and further preferably not lower than 0.5% by mass and not higher than 5% by mass.

<3. Skin External Agent>

A skin external agent according to the embodiment contains the MMP-1 expression suppression agent described above. Here, “containing the MMP-1 expression suppression agent” may denote that the skin external agent partially contains the MMP-1 expression suppression agent. That is, the skin external agent may include the MMP-1 expression suppression agent and another component. Further, the skin external agent may be composed only of the MMP-1 expression suppression agent described above. That is, the MMP-1 expression suppression agent itself may be used as the skin external agent.

Here, the “skin external agent” may mean a skin care agent in a broad sense, may be for beauty usage, or may be for treatment usage. The skin external agent is suitably used as a cosmetic, a quasi drug, or a medicine, for example. The skin external agent can contain an active ingredient and a base. The active ingredient can be at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop. The base can be a fat and oil base, a water-soluble base, an emulsion base, or a lotion base, for example. The skin external agent for beauty usage can be used as a skin lotion, an emulsion, a beauty essence (including emulsion type), or a cream, for example.

<3. First Experiment>

A first experiment was performed in order to search for a plant extract (Chinese medicine extract) that has an anti-MMP-1 action. In the first experiment, a plurality of plant extracts were used. The plurality of plant extracts used in the first experiment were an extract of Saxifraga sarmentosa, an extract of rosemary, an extract of licorice, an extract of Melissa officinalis, an extract of wild thyme, an extract of hop, an extract of linden, and an extract of Artemisia leaf (extract of Artemisia princeps).

As the extract of Saxifraga sarmentosa, “Saxifraga sarmentosa extraction liquid” manufactured by MARUZEN PHARMACEUTICALS CO., LTD. was used. “Saxifraga sarmentosa extraction liquid” manufactured by MARUZEN PHARMACEUTICALS CO., LTD. is an extract extracted from the entire plant of Saxifraga sarmentosa. As the extract of rosemary, “rosemary extraction liquid-J” manufactured by MARUZEN PHARMACEUTICALS CO., LTD. was used. “Rosemary extraction liquid-J” manufactured by MARUZEN PHARMACEUTICALS CO., LTD. is an extract extracted from the leaf of rosemary.

As the extract of licorice, “licorice extraction liquid” manufactured by MARUZEN PHARMACEUTICALS CO., LTD. was used. “Licorice extraction liquid” manufactured by MARUZEN PHARMACEUTICALS CO., LTD. is an extract extracted from the root of licorice. It should be noted that “root” may include rhizome. As the extract of Melissa officinalis, “Melissa officinalis extraction liquid-J” manufactured by MARUZEN PHARMACEUTICALS CO., LTD. was used. “Melissa officinalis extraction liquid-J” manufactured by MARUZEN PHARMACEUTICALS CO., LTD. is an extract extracted from the leaf of Melissa officinalis (sweet balm). As the extract of wild thyme, “wild thyme extraction liquid” manufactured by MARUZEN PHARMACEUTICALS CO., LTD. was used. “Wild thyme extraction liquid” manufactured by MARUZEN PHARMACEUTICALS CO., LTD. is an extract extracted from the above-ground part of wild thyme (Thymus serpyllum). As the extract of hop, “hop extraction liquid” manufactured by MARUZEN PHARMACEUTICALS CO., LTD. was used. “Hop extraction liquid” manufactured by MARUZEN PHARMACEUTICALS CO., LTD. is an extract extracted from the flower of hop.

As the extract of linden, “linden extraction liquid” manufactured by MARUZEN PHARMACEUTICALS CO., LTD. was used. “Linden extraction liquid” manufactured by MARUZEN PHARMACEUTICALS CO., LTD. is an extract extracted from the flower of Tilia cordata. Artemisia princeps is a plant of genus Artemisia of family Asteraceae, and is a perennial herb. A herbal medicine using the leaf of Artemisia princeps is referred to as Artemisia leaf. As the extract of Artemisia princeps (extract of Artemisia leaf), “Artemisia leaf extraction liquid” manufactured by MARUZEN PHARMACEUTICALS CO., LTD. was used. “Artemisia leaf extraction liquid” manufactured by MARUZEN PHARMACEUTICALS CO., LTD. is an extract extracted from the leaf of Artemisia princeps.

These extracts are also used in a second experiment and experiments thereafter, and Examples.

In the first experiment, each of a plurality of plant extracts was 100-fold diluted with a phosphate-buffered saline (PBS(−)). 50 μL of the diluted plant extract was added to HaCaT, and the resultant matter was incubated for one hour and then was subjected to UVA irradiation. The UVA amount was 16 J, the irradiation time was 13 minutes and 18 seconds, and the irradiation distance was 20 cm. After 24 hours of the irradiation, cells were collected and subjected to qPCR. Then, influence of each plant extract on MMP-1 expression was examined.

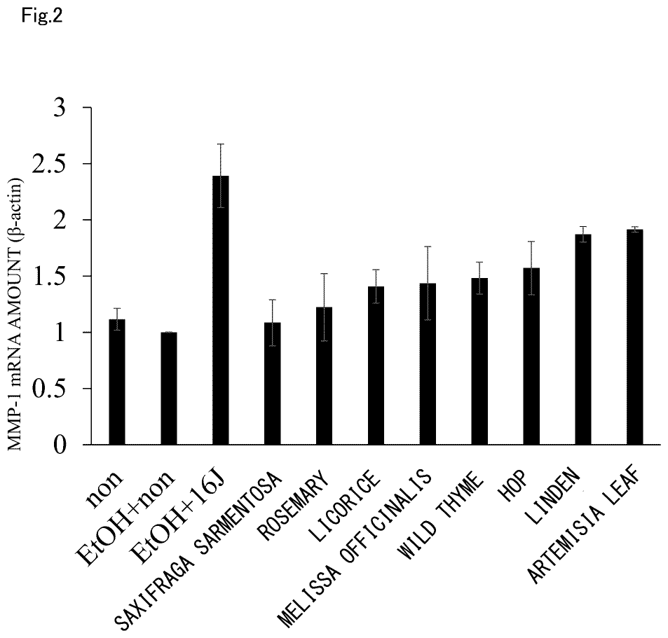

FIG. 2 shows results of the first experiment. The graph shown in FIG. 2 indicates the MMP-1 expression amount obtained when the MMP-1 expression amount of a group (without UVA irradiation) in which ethanol (EtOH) was added to HaCaT is defined as 1. In FIG. 2, “EtOH+non” indicates a group (without UVA irradiation) in which ethanol (EtOH) was added to HaCaT. In FIG. 2, “non” indicates a group (without UVA irradiation) in which ethanol was not added to HaCaT. In FIG. 2, “EtOH+16 J” indicates a group in which ethanol (EtOH) was added to HaCaT and which was subjected to 16 J UVA irradiation. In “non”, “EtOH+non”, and “EtOH+16 J”, the plant extract was not added. In FIG. 2, “Saxifraga sarmentosa”, “rosemary”, “licorice”, “Melissa officinalis”, “wild thyme”, “hop”, “linden”, and “Artemisia leaf” indicate groups in which extracts thereof were respectively added and which were subjected to 16 J UVA irradiation.

As shown in FIG. 2, the MMP-1 expression amount of “EtOH+16 J” is about 2.4 times the MMP-1 expression amount of “EtOH+non”, and has been greatly increased. In contrast to this, when each plant extract was added, the MMP-1 expression amount was suppressed. In particular, in “Saxifraga sarmentosa”, “rosemary”, “licorice”, “Melissa officinalis”, “wild thyme”, and “hop”, sufficient suppression of the MMP-1 expression amount was observed when compared with that of “EtOH+16 J”. In “Saxifraga sarmentosa”, “rosemary”, “licorice”, “Melissa officinalis”, “wild thyme”, and “hop”, the MMP-1 expression amount is not greater than about 1.8 times that of “EtOH+non”. Therefore, “Saxifraga sarmentosa”, “rosemary”, “licorice”, “Melissa officinalis”, “wild thyme”, and “hop” are each suitable as an active ingredient of the MMP-1 expression suppression agent.

In particular, in “Saxifraga sarmentosa” and “rosemary”, the MMP-1 expression amount is not greater than a half of that of “EtOH+16 J”, and thus, “Saxifraga sarmentosa” and “rosemary” are understood to be particularly suitable. In “Saxifraga sarmentosa” and “rosemary”, the MMP-1 expression amount is not greater than about 1.2 times that of “EtOH+non”, and the influence of UVA is very small.

<4. Second Experiment>

In the second experiment, effects of the plant extracts on mice were examined. In the second experiment, out of “Saxifraga sarmentosa”, “rosemary”, “licorice”, “Melissa officinalis”, “wild thyme”, and “hop”, “Saxifraga sarmentosa” in which the MMP-1 expression amount suppression action was the largest in the first experiment, and “hop” in which the MMP-1 expression amount suppression action was the smallest in the first experiment were used.

In the second experiment, hair on the backs of WT (Wild Type) mice of 15 weeks old was sheared, 100 μL of 4% sodium dodecyl sulfate (SDS) was applied to the mice, and the mice were subjected to drying for 30 minutes. Then, the extract of Saxifraga sarmentosa and the extract of hop that were 10-fold diluted, and ethanol as a control were applied in an amount of 100 μL, respectively, and UVA irradiation was performed for two weeks. The UVA irradiation was performed, in an irradiation amount of 30 J per radiation, a total of seven times in two weeks, that is, a total of 210 J of irradiation was performed. Then, a tissue lysate was prepared from a skin tissue of each mouse, and MMP-1 expression was examined by western-blotting.

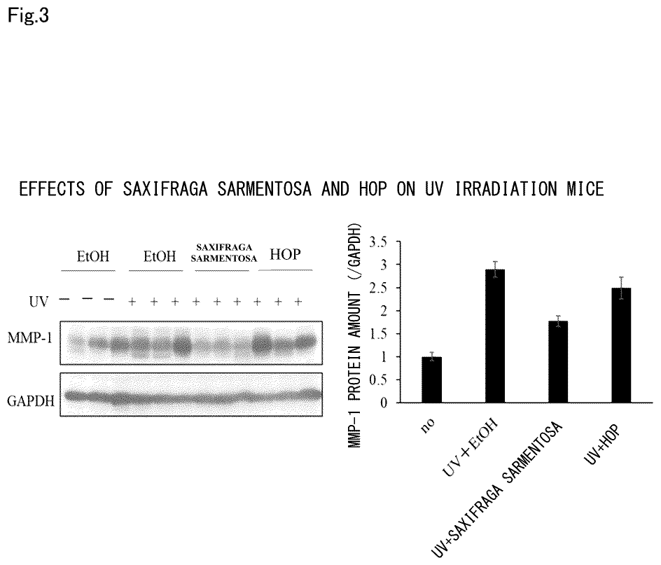

FIG. 3 shows results of the second experiment. The graph in FIG. 3 indicates the MMP-1 expression amount obtained when the MMP-1 expression amount of a UVA non-irradiation mouse is defined as 1. In FIG. 3, “no”, indicates a UVA non-irradiation mouse. In FIG. 3, “UV+EtOH” indicates a mouse to which ethanol was applied as a control and which was subjected to UVA irradiation, “UV+Saxifraga sarmentosa” indicates a mouse to which the extract of Saxifraga sarmentosa was applied and which was subjected to UVA irradiation, and “UV+hop” indicates a mouse to which the extract of hop was applied and which was subjected to UVA irradiation.

As shown in FIG. 3, the MMP-1 expression amount of “UV+EtOH” is about 3 times the MMP-1 expression amount of “no”, and has been greatly increased. In contrast to this, the MMP-1 expression amount of each of “UV+Saxifraga sarmentosa” and “UV+hop” has been sufficiently decreased when compared with that of “UV+EtOH”. Therefore, it was confirmed that, also in mice, Saxifraga sarmentosa and hop are effective in MMP-1 expression suppression. Out of “Saxifraga sarmentosa”, “rosemary”, “licorice”, “Melissa officinalis”, “wild thyme”, and “hop”, in even “hop” in which the MMP-1 expression amount suppression action was the smallest in the first experiment, the MMP-1 expression suppression action in mice was confirmed. Therefore, it is understood that the MMP-1 expression suppression action in mice is obtained also in “rosemary”, “licorice”, “Melissa officinalis”, and “wild thyme”.

<5. Third Experiment>

In a third experiment, similar to the second experiment, mice to which the extract of Saxifraga sarmentosa and the extract of hop, and ethanol as a control were respectively applied and which were subjected to UVA irradiation were used. After the UVA irradiation, a tissue lysate was prepared from a skin tissue of each mouse, and phosphorylation of PKCδ was examined by western-blotting.

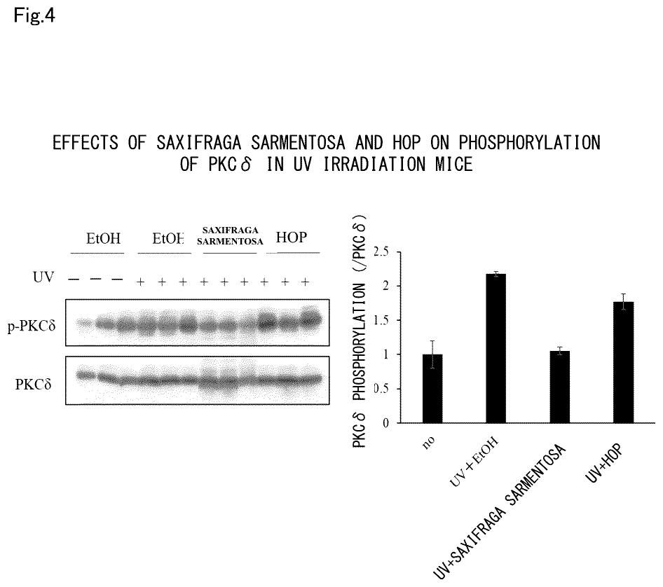

FIG. 4 shows results of the third experiment. The graph in FIG. 4 indicates the PKC phosphorylation amount obtained when the PKCδ phosphorylation amount of a UVA non-irradiation mouse is defined as 1. In FIG. 4, “no” indicates a UVA non-irradiation mouse. In FIG. 4, “UV+EtOH” indicates a mouse to which ethanol is applied as a control and which was subjected to UVA irradiation, “UV+Saxifraga sarmentosa” indicates a mouse to which the extract of Saxifraga sarmentosa was applied and which was subjected to UVA irradiation, and “UV+hop” indicates a mouse to which the extract of hop was applied and which was subjected to UVA irradiation.

As shown in FIG. 4, the PKCδ phosphorylation amount of “UV+EtOH” is about 2.2 times the PKCδ phosphorylation amount of “no”, and has been greatly increased. In contrast to this, the PKCδ phosphorylation amount of each of “UV+Saxifraga sarmentosa” and “UV+hop” has been sufficiently decreased when compared with that of “UV+EtOH”. Therefore, Saxifraga sarmentosa and hop each suppress phosphorylation of PKC6. From this, it was revealed that Saxifraga sarmentosa and hop each inhibit the activity of PKC6, and each act as a PKCδ inhibitor. That is, Saxifraga sarmentosa and hop each suppress expression of MMP-1 through inhibition of the activity of PKGS. These results indicate that rosemary, licorice, Melissa officinalis, wild thyme, and the like also have a similar action mechanism.

<6. Fourth Experiment>

In a fourth experiment, effects, on the skin of humans, of the skin external agent containing a plant extract were examined. In the fourth experiment, as the plant extract, “Saxifraga sarmentosa” was used. The condition for the fourth experiment is as follows. Subject: eleven women from 34 years old to 62 years old (average age: 50.1 years old, n=11).

(two persons in their thirties, two persons in their forties, five persons in their fifties, and two persons in their sixties).

### Control Sample

First control sample: DSR Corporation “NMF moisturizer B” (skin lotion)
Second control sample: DSR Corporation “Sebus cream WS” (cream)

### Test Sample

First test sample: The first control sample added with the Saxifraga sarmentosa extract by 3%
Second test sample: The second control sample added with the Saxifraga sarmentosa extract by 3%
   Test method: Before performing the test, a skin replica at the depth of the outer corner of each of the right eye and the left eye of each subject was collected. Then, for two weeks, twice per day (morning and night), the first and second control samples were applied to the outer corner of the left eye of the subject and the first and second test samples were applied to the outer corner of the right eye. Makeup other than applying the control sample and the test sample was not limited. After two weeks, a skin replica at the outer corner of each of the right eye and the left eye of each subject was collected again.
   Evaluation method: For each replica sheet, three points were randomly extracted, and the wrinkle area and the maximum wrinkle depth were measured. Then, the measurement results of the eleven persons were averaged. Changes in the wrinkle area and the maximum wrinkle depth before and after the test are indicated in terms of proportion (%) obtained when those before the test are defined as 100.

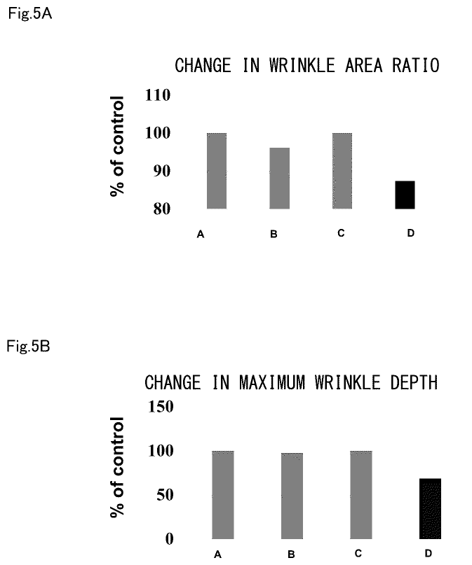

FIG. 5 shows results of the fourth experiment. FIG. 5A shows the change rate of the wrinkle area before and after the test, and FIG. 5B shows the change rate of the maximum wrinkle depth. In FIG. 5A and FIG. 5B, A indicates the average (n=11) in a skin sample at the outer corner of the left eye before the test, and B indicates the average (n=11) in a skin sample after the control sample was applied for two weeks, C indicates the average (n=11) in a skin sample at the outer corner of the right eye before the test, and D indicates the average (n=11) in a skin sample after the test sample was applied for two weeks.

As shown in the fourth experiment, as a result of application of the skin lotion and the cream (test sample) containing the Saxifraga sarmentosa extract for two weeks, the test sample was confirmed to have a tendency of improving wrinkles when compared with the control sample. Specifically, at the outer corner of the left eye to which the control sample was applied, the wrinkle area ratio after the test was 96.1% (see B in FIG. 5A), and the maximum wrinkle depth was 97.5% (see B in FIG. 5B). These were slight improvements. In contrast to this, at the outer corner of the right eye to which the test sample was applied, the wrinkle area ratio was 87.3% (see D in FIG. 5A) and thus a large improvement of 12.7% was obtained, and the maximum wrinkle depth was 68.5% (see D in FIG. 5B) and thus a large improvement of 31.5% was obtained.

### EXAMPLES

Cosmetics (skin external agent) having the following compositions were produced as the MMP-1 expression suppression agent. “Plant extract” in each Example is one of the extract of Saxifraga sarmentosa, the extract of rosemary, the extract of licorice, the extract of Melissa officinalis, the extract of wild thyme, and the extract of hop. A total of six types which respectively use different extracts were produced in each of Examples below. It should be noted that the present invention is not limited to Examples below.

First Example (skin lotion A)

| Component name | Combination (% by mass) |
|---|---|
| plant extract | 1.000 |
| glycerin | 3.000 |
| propanediol | 3.000 |
| PCA-Na | 0.200 |
| serine | 0.032 |
| glycine | 0.022 |
| glutamic acid | 0.017 |
| alanine | 0.009 |
| lysine | 0.007 |
| lysine HCl | 0.003 |
| arginine | 0.008 |
| threonine | 0.005 |
| proline | 0.003 |
| leucine | 0.002 |
| histidine HCl | 0.002 |
| valine | 0.002 |
| sodium aspartate | 0.001 |
| isoleucine | 0.001 |
| phenylalanine | 0.001 |
| allantoin | 0.001 |
| sodium hyaluronate | 0.030 |
| ethylhexylglycerin | 0.200 |
| betaine | 0.600 |
| sorbitol | 0.084 |
| taurine | 0.027 |
| xanthan gum | 0.003 |
| carbomer | 0.200 |
| phenoxyethanol | 0.500 |
| water | remainder |
| total | 100.000 |

Second Example (skin lotion B)

| Component name | Combination (% by mass) |
|---|---|
| plant extract | 1.000 |
| glycerin | 3.000 |
| propanediol | 3.000 |
| sodium ascorbyl phosphate | 1.000 |
| PCA-Na | 0.200 |
| serine | 0.032 |
| glycine | 0.022 |
| glutamic acid | 0.017 |
| alanine | 0.009 |
| lysine | 0.007 |
| lysine HCl | 0.003 |
| arginine | 0.008 |
| threonine | 0.005 |
| proline | 0.003 |
| leucine | 0.002 |
| histidine HCl | 0.002 |
| valine | 0.002 |
| sodium aspartate | 0.001 |
| isoleucine | 0.001 |
| phenylalanine | 0.001 |
| allantoin | 0.001 |
| sodium hyaluronate | 0.030 |
| ethylhexylglycerin | 0.200 |
| betaine | 0.600 |
| sorbitol | 0.084 |
| taurine | 0.027 |
| xanthan gum | 0.003 |
| carbomer | 0.200 |
| sodium citrate | 0.050 |
| phenoxyethanol | 0.500 |
| water | remainder |
| total | 100.000 |

Third Example (emulsion)

| Component name | Combination (% by mass) |
|---|---|
| plant extract | 1.000 |
| octyldodecanol | 10.000 |
| glycerin | 4.000 |
| propanediol | 4.000 |
| sorbitan monostearate | 1.000 |
| polyoxyethylene hydrogenated castor oil | 0.800 |
| cetanol | 0.300 |
| stearyl alcohol | 0.200 |
| methylparaben | 0.150 |
| potassium hydroxide | 0.100 |
| carbomer | 0.250 |
| sodium citrate | 0.050 |
| perfume | 0.100 |
| water | remainder |
| total | 100.000 |

Fourth Example (beauty essence A)

| | Combination |
|---|---|
| Component name | (% by mass) |
| plant extract | 1.000 |
| glycerin | 3.000 |
| propanediol | 3.000 |
| 1,3-butylene glycol | 1.000 |
| PCA-Na | 0.200 |
| serine | 0.032 |
| glycine | 0.022 |
| glutamic acid | 0.017 |
| alanine | 0.009 |
| lysine | 0.007 |
| arginine | 0.008 |
| threonine | 0.005 |
| proline | 0.003 |
| sodium hyaluronate | 0.030 |
| ethylhexylglycerin | 0.200 |
| betaine | 0.600 |
| sorbitol | 0.084 |
| xanthan gum | 0.003 |
| carbomer | 0.200 |
| acrylates/beheneth-25 methacrylate copolymer | 0.400 |
| potassium hydroxide | 0.060 |
| sodium citrate | 0.050 |
| phenoxyethanol | 0.500 |
| water | remainder |
| total | 100.000 |

Fifth Example (beauty essence B: emulsion type)

| | Combination |
|---|---|
| Component name | (% by mass) |
| plant extract | 1.000 |
| glycerin | 3.000 |
| propanediol | 3.000 |
| 1,3-butylene glycol | 1.000 |
| ceramide 1 | 0.00003 |
| ceramide 2 | 0.035 |
| ceramide 3 | 0.100 |
| ceramide 5 | 0.005 |
| ceramide 6II | 0.030 |
| phytosphingosine | 0.025 |
| cholesterol | 0.055 |
| phytosterols | 0.020 |
| PCA-Na | 0.200 |
| serine | 0.032 |
| glycine | 0.022 |
| glutamic acid | 0.017 |
| alanine | 0.009 |
| lysine | 0.007 |
| arginine | 0.008 |
| threonine | 0.005 |
| proline | 0.003 |
| sodium hyaluronate | 0.030 |
| ethylhexylglycerin | 0.200 |
| betaine | 0.600 |
| sorbitol | 0.084 |
| xanthan gum | 0.130 |
| carbomer | 0.100 |
| acrylates/beheneth-25 methacrylate copolymer | 0.400 |
| sodium citrate | 0.050 |
| potassium hydroxide | 0.060 |
| phenoxyethanol | 0.500 |
| water | remainder |
| total | 100.000 |

Sixth Example (cream A)

| Component name | Combination (% by mass) |
|---|---|
| plant extract | 1.000 |
| squalane | 5.000 |
| glycerin | 3.000 |
| propanediol | 3.000 |
| shea butter | 2.000 |
| jojoba seed oil | 2.000 |
| macadamia ternifolia seed oil | 1.000 |
| octyldodecanol | 1.000 |
| meadowfoam seed oil | 0.500 |
| PCA-Na | 0.200 |
| serine | 0.032 |
| glycine | 0.022 |
| glutamic acid | 0.017 |
| alanine | 0.009 |
| lysine | 0.007 |
| arginine | 0.008 |
| threonine | 0.005 |
| proline | 0.003 |
| sodium hyaluronate | 0.030 |
| ethylhexylglycerin | 0.200 |
| betaine | 0.600 |
| sorbitol | 0.084 |
| xanthan gum | 0.600 |
| agar | 0.400 |
| carbomer | 0.300 |
| tocopherol | 0.100 |
| sodium citrate | 0.050 |
| potassium hydroxide | 0.060 |
| phenoxyethanol | 0.500 |
| water | remainder |
| total | 100.000 |

Seventh Example (cream B)

| | Combination |
|---|---|
| Component name | (% by mass) |
| plant extract | 1.000 |
| triethylhexanoin | 20.000 |
| jojoba seed oil | 10.000 |
| glycerin | 5.000 |
| propanediol | 5.000 |
| dipentaerythrityl hexahydroxystearate/hexastearate/ | 1.000 |
| hexarosinate | |
| PEG-240/HDI copolymer bis-decyltetradeceth-20 ether | 1.000 |
| polysorbate 60 | 0.500 |
| acrylates/C10-30 alkyl acrylate crosspolymer | 0.050 |
| potassium hydroxide | 0.100 |
| phenoxyethanol | 0.500 |
| water | remainder |
| total | 100.000 |

1. A matrix metalloproteinase 1 expression suppression agent containing, as an active ingredient,
at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop.

2. The matrix metalloproteinase 1 expression suppression agent, according to claim 1, that suppresses expression of matrix metalloproteinase 1, through inhibition of activity of protein kinase C-delta.

3. A skin external agent containing the matrix metalloproteinase 1 expression suppression agent according to claim 1 or 2.

4. Use, for matrix metalloproteinase 1 expression suppression, of at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop.

5. The use according to claim 4, wherein
expression of matrix metalloproteinase 1 is suppressed through inhibition of activity of protein kinase C-delta.

6. Use, for production of a skin external agent for matrix metalloproteinase 1 expression suppression, of at least one selected from the group consisting of Saxifraga sarmentosa, an extract of Saxifraga sarmentosa, rosemary, an extract of rosemary, licorice, an extract of licorice, Melissa officinalis, an extract of Melissa officinalis, wild thyme, an extract of wild thyme, hop, and an extract of hop.

7. The use according to claim 4, wherein
the skin external agent is for suppressing expression of matrix metalloproteinase 1, through inhibition of activity of protein kinase C-delta.

8. A method comprising:
